(19) United States
(12) Patent Application Publication
(10) Pub. No.: US 2022/0183882 A1
(43) Pub. Date: Jun. 16, 2022

(54) DEVICES AND METHODS FOR TREATING AQUEOUS COLLECTOR CHANNELS OF AN EYE TO REDUCE INTRAOCULAR PRESSURE

(71) Applicant: The Regents of the University of California, Oakland, CA (US)

(72) Inventors: Sameh Mosaed, Laguna Hills, CA (US); Ken Y. Lin, Irvine, CA (US)

(21) Appl. No.: 17/542,392

(22) Filed: Dec. 4, 2021

Related U.S. Application Data
(60) Provisional application No. 63/123,962, filed on Dec. 10, 2020.

(51) Int. Cl. A61F 9/007; A61F 9/008; G16H 20/40

(52) U.S. Cl. CPC A61F 9/00772; A61F 9/00821; A61B 2018/00994; A61F 2009/00891; A61F 2009/00868; G16H 20/40

(57) ABSTRACT

Devices and method for treating aqueous collector channels or tissue(s) associated with aqueous collector channels to improve or enhance outflow of aqueous humor from an eye thereby reducing intraocular pressure in the eye.

### RELATED APPLICATION

This patent application claims priority to copending U.S. Provisional Patent Application No. 63/123,962 entitled Devices and Methods for Treating Aqueous Collector Channels of an Eye to Reduce lntraoccular Pressure, filed Dec. 10, 2020, the entire disclosure of whish it expressly incorporated herein by reference.

### FIELD OF THE INVENTION

The present disclosure pertains generally to the fields of biomedical engineering and surgery and more particularly to devices and methods treating aqueous collector channels of a mammalian eye to improve outflow of aqueous humor and reduce intraocular pressure (IOP).

### BACKGROUND OF THE INVENTION

Pursuant to 37 CFR 1.71(e), this patent document contains material which is subject to copyright protection and the owner of this patent document reserves all copyright rights whatsoever.

A liquid substance called aqueous humor is produced within the eye and collects within the anterior chamber of the eye. Excess aqueous humor naturally drains out of the anterior chamber, through permeable tissue known as trabecular meshwork and into an annular canal known as Schlemm's Canal. After entering Schlemm's canal, the excess aqueous humor drains outwardly through a network of aqueous collector channels which empty into the venous system.

In some types of glaucoma, the outflow or drainage of aqueous humor is inadequate, which in turn causes abnormally elevated pressure within the eye (i.e., intraocular pressure). Over time, such elevated intraocular pressure can cause damage to the retinal and/or optic nerve, with resultant loss of sight.

A number of pharmacologic and surgical treatments have been developed to improve aqueous outflow and reduce elevated intraocular pressure. Among these are a number of minimally invasive glaucoma surgery (MIGS) procedures, which include surgeries to incise or form opening(s) in the trabecular meshwork as well as implantation of tubes or stents to facilitate aqueous drainage from aqueous humor from the anterior chamber, through the trabecular meshwork, and into Schlemm's canal. However, once aqueous humor has entered Schlemm's Canal, it must then drain from Schlemm's Canal, though the aqueous collector channels and into the subject's venous circulation. If the ostia or orifices of collector channels tend to collapse or close, or if the collector channels themselves are obstructed or incompetent, the objective of improving aqueous drainage and lowering intraocular pressure may not be fully realized.

Therefore, there exists a need for the development of new devices and a methods to improve or increase drainage of aqueous humor into and through collector channels downstream of Schlemm's Canal.

### SUMMARY OF THE INVENTIONS

Disclosed herein are methods, devices and systems useable to improve or increase drainage of aqueous humor from Schlemm's Canal, into and through the aqueous collector channels, thereby facilitating successful reduction of abnormally elevated intraocular pressure.

In accordance with one aspect of the disclosure, there is provided a system useable for treating a target aqueous collector channel in an eye of a subject to increase outflow of aqueous humor through that target collector channel, said system comprising: a first device or system useable to locate the target collector channel; a second device or system useable to form an opening in trabecular meshwork tissue which overlies or provides access to a portion of a Schlemm's Canal of the eye in which an orifice or ostium of the target collector channel is located; and a third device or system useable to treat tissue in or near the target collector channel to improve patency, openness, size, flow capacity or competency that target collector channel and/or an orifice or ostium associated with that target collector channel. In some embodiments, each of said first, second and third devices or systems may comprise a separate instrument. In some embodiments, two or more of said first, second and third devices or systems may comprise or be combined in a single instrument. In some embodiments, the first device or system may comprise a scope, viewing device, imaging apparatus, microendoscope, laser microendoscope or other apparatus operable to view, image, determine size, patency or flow capacity of, and/or otherwise locate target aqueous collector channel(s) with or without prior introduction of a contrast medium. In some embodiments, the second device or system may comprises a knife, dual blade knife, goniotomy knife, goniotome, cutting apparatus, ablating apparatus, electrosurgical apparatus, cautery, bipolar probe, energy emitting probe, laser probe, TrabEx™ device, TrabEx+™ device, Trabectome® device, Kahook Dual Blade® (KDB or KDB Glide) device, or other suitable apparatus useable to incise, sever, ablate, tear, remove or otherwise form said one or more opening(s) in trabecular meshwork tissue which overlies or is located near target collector channel(s). In some embodiments, the third device or system may comprise an energy-emitting probe, laser, tissue warming or heating device, coagulation device, or other apparatus operable to photocoagulate, heat, thermally modify, optically modify or otherwise treat tissue in or near target collector channel(s) to improve patency, openness, size, flow capacity or competency of target collector channel(s) and/or orifice(s) or ostia associated with target collector channel(s). In some embodiments, each of the first device or system, second device or system and third device or system comprises a separate instrument or entity. In some embodiments, at least two of said first device or system, second device or system and third device or system are combined or integrated, in whole or in part, into a single instrument or entity. In some embodiments, all of said first device or system, second device or system and third device or system are combined or integrated, in whole or in part, into a single instrument or entity. In some embodiments, the third device or system may comprises a laser of a type selected from; argon, krypton, diode or frequency doubled ND: YAG lasers. In some embodiments, the third device or system may deliver argon laser energy at 1 watt, non-pulsed, for a duration of 0.5 to 1 second, with a spot size of 100 microns. In some embodiments, the third device or system may comprise an apparatus for aiming, aligning or positioning that device or system relative to an orifice or ostium or a target collector channel such as, for example, a scope or imaging device useable to visualize the orifice or ostium of a target collector channel and/or one or more rods or members which advance or insert into an orifice or ostium of a target collector channel and, optionally, may deliver laser energy or another form of energy through or about a periphery of said one or more rods or members which advance or insert into an orifice or ostium of a target collector channel to effect the desired treatment. In some embodiments which have said extendable rods or members, the apparatus for aiming, aligning or positioning may further comprise a head member which is positionable in a Schlemm's Canal of the eye and the plurality of said extendable rods or members may be configured to extend from said head member when said head member is positioned in Schlemm's Canal, such that those extendable rods or members which are aligned with an orifice or ostium of a collector channel will extend further than others of said extendable rods or members which are not so aligned and the system may be further configured to selectively deliver laser energy or other treatment through, or in an area around or near, those extendable rods or members which have extended further than others of said extendable rods or members, thereby selectively delivering laser energy or other treatment to or through an orifice or ostium of a collector channel into which those extendable rods or members have advanced.

Further in accordance with the present disclosure, there is provided a method for improving or stabilizing drainage of aqueous humor from an anterior chamber of an eye of a subject, said method comprising the steps of: a) locating one or more target aqueous collector channels; b) forming one or more opening(s) in trabecular meshwork tissue to un-roof one or more region(s) of Schlemm's Canal of the eye in a manner which exposes or allows access to the target collector channel(s); and c) treating the orifice or ostium of each target collector channel and/or an area around or near the orifice or ostium and/or a wall or portion of a wall of the target collector channel in a manner that increases outflow of aqueous humor from an anterior chamber of the eye through the target collector channel. In some embodiments, the step of locating one or more target collector channel(s) may comprise: introducing a contrast agent into the eye such that it outflows through aqueous collector channels; obtaining image data indicating or showing flow of contrast agent through collector channels; and using the obtained image data to identify or select the target collector channel(s). In such embodiments, the step of obtaining image data may comprise using a laser microendoscope in either an ab interno or ab externo manner as described in herein incorporated United States Patent Application Publication No. 2016/0262606 entitled Device, System and Method for Functional Imaging of Episcleral Vessels (Mosaed et al.) and/or the step of using the obtained image data to identify or select the target collector channel(s) comprises using a vessel segmentation algorithm in a manner described in herein incorporated United States Patent Application Publication No. 2016/0262606 entitled Device, System and Method for Functional Imaging of Episcleral Vessels (Mosaed et al.). In some embodiments, the step of forming one or more opening(s) in trabecular meshwork tissue to un-roof one or more region(s) of Schlemm's Canal of the eye in a manner which exposes or allows access to the target collector channel(s) may comprise: using a knife, dual blade knife, goniotomy knife, goniotome, cutting apparatus, ablating apparatus, electrosurgical apparatus, cautery, bipolar probe, energy emitting probe, laser probe, TrabEx™ device, TrabEx+™ device, Trabectome® device, Kahook Dual Blade® (KDB or KDB Glide) device, or other suitable apparatus to incise, sever, ablate, tear, remove, cut a strip from, or otherwise form said one or more opening(s) in the trabecular meshwork tissue. In some embodiments, the step of treating the orifice or ostium of each target collector channel and/or an area around or near the orifice or ostium and/or a wall or portion of a wall of the target collector channel in a manner that increases outflow of aqueous humor from an anterior chamber of the eye through the target collector channel may comprise: delivering laser or other energy to photocoagulate, stiffen, reduce flexibility of, reduce elasticity of, or otherwise modify the orifice or ostium and/or tissue around or near the orifice of ostium and/or a wall or portion of a wall of the target collector channel in a manner that reduces likelihood of collapse or close of, maintain patency of and/or improves flow through the orifice or ostium and/or the collector channel. In some embodiments, a first device or system is used to locate said target collector channel(s); a second device or system is used to form said one or more opening(s) in trabecular meshwork tissue to un-roof one or more region(s) of Schlemm's Canal of the eye in a manner which exposes or allows access to the target collector channel(s); and a third device or system is used to treat the orifice or ostium of each target collector channel and/or an area around or near the orifice or ostium and/or a wall or portion of a wall of the target collector channel in a manner that increases outflow of aqueous humor from an anterior chamber of the eye through the target collector channel. In some such embodiments, the second and third devices or systems may be combined, in whole or in part, into a single instrument or entity that is insertable into the eye and useable to a) form said form one or more opening(s) in trabecular meshwork tissue and b) treat the orifice or ostium of each target collector channel and/or an area around or near the orifice or ostium and/or a wall or portion of a wall of the target collector channel in a manner that increases outflow of aqueous humor from an anterior chamber of the eye through the target collector channel. Such single instrument or entity may, for example, deliver a first laser treatment to form said form one or more opening(s) in trabecular meshwork tissue and a second laser treatment to treat the orifice or ostium of each target collector channel and/or an area around or near the orifice or ostium and/or a wall or portion of a wall of the target collector channel in a manner that increases outflow of aqueous humor from an anterior chamber of the eye through the target collector channel. The first laser treatment may comprise, for example, excimer laser energy and the second laser treatment may comprise, for example, argon laser energy. In other embodiments, all of the first, second and third devices or systems may be combined, in whole or in part, into a single instrument or entity that is insertable into the anterior chamber of the eye and useable to a) locate target collector channel(s), b) form said form one or more opening(s) in trabecular meshwork tissue and c) treat the orifice or ostium of each target collector channel and/or an area around or near the orifice or ostium and/or a wall or portion of a wall of the target collector channel in a manner that increases outflow of aqueous humor from an anterior chamber of the eye through the target collector channel such that the single instrument or entity is useable to; i) obtain image data indicating or showing flow of contrast agent through collector channels, ii) deliver a first laser treatment (e.g., excimer laser) to form said form one or more opening(s) in trabecular meshwork tissue, and iii) deliver a second laser treatment (e.g., argon laser) to treat the orifice or ostium of each target collector channel and/or an area around or near the orifice or ostium and/or a wall or portion of a wall of the target collector channel in a manner that increases outflow of aqueous humor from an anterior chamber of the eye through the target collector channel.

Any feature or combination of features described herein are included within the scope of the present disclosure provided that the features included in any such combination are not mutually inconsistent as will be apparent from the context, this specification, and the knowledge of one of ordinary skill in the art. Additional advantages and aspects of the present disclosure are apparent in the following detailed description and claims.

Still further aspects and details of the present disclosure will be understood upon reading of the detailed description and examples set forth herebelow.

### BRIEF DESCRIPTION OF THE DRAWINGS

The following figures are included in this patent application and referenced in the following Detailed Description. These figures are intended only to illustrate certain aspects or embodiments of the present disclosure and do not limit the scope of the present disclosure in any way:

FIG. 1 is a lateral perspective view of an intact human eye.

FIG. 1A is a transverse sectional view of a human eye.

FIG. 1B is a schematic diagram representing anatomical structures involved in the outflow and drainage of aqueous humor from a human eye.

FIG. 2 is a transverse sectional view of a human eye with one embodiment of a microendoscope positioned within a scleral flap incision while being used to locate target collector channel(s) for subsequent treatment.

FIG. 3 is a transverse sectional view of a human eye with an instrument being used to un-roof a segment of Schlemm's Canal to expose or provide access to target collector channel(s).

FIG. 3A is a schematic diagram representing anatomical structures involved in the outflow and drainage of aqueous humor from a human eye after a segment of Schlemm's Canal has been un-roofed as shown in FIG. 3.

FIG. 4 is a transverse sectional view of a human eye with one embodiment of a laser treatment system being used to deliver laser treatment to a target collector channel.

FIG. 4A is a schematic diagram representing anatomical structures involved in the outflow and drainage of aqueous humor from a human eye with

FIG. 4 is a transverse sectional view of a human eye with a laser treatment system being used to deliver laser treatment to a target collector channel as shown in FIG. 3.

FIG. 5 is a transverse sectional view of a human eye with one embodiment of a laser device positioned within the eye.

FIG. 6 is a longitudinal sectional view of a distal portion of the laser device shown in FIG. 5.

FIG. 7A is a diagram showing a distal portion of another embodiment of a laser treatment device useable for delivery of laser treatment to a target collector channel.

FIG. 7B is an enlarged view of the distal portion of the device shown in FIG. 7A.

### DETAILED DESCRIPTION OF THE INVENTION

The following detailed description and the accompanying drawings are intended to describe and illustrate some, but not necessarily all, examples or embodiments of the present disclosure. The described examples or embodiments are to be considered in all respects as illustrative but not restrictive. The contents of this detailed description and the accompanying drawings do not limit the scope of this disclosure in any way.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, he articles “a” and “an” are used herein to refer to one or to more than one (i.e., to at least one) of the grammatical object of the article. By way of example, “an element” means one element or more than one element.

In the accompanying drawings, certain anatomical structures are labeled as follows:
Eye: E
Cornea: C
Limbus: LI
Sclera: S
Lens: L
Anterior Chamber: AC
Iridocorneal Angle: A
Schlemm's Canal: SC
Trabecular Meshwork: TM
Collector Channel: CC
Orifice or Ostium of Collector Channel: OS
Episcleral Vein: EV

The present disclosure includes a method for treating an aqueous collector channel and/or for treating an orifice or ostium of an aqueous collector channel and/or for treating an area around or near an orifice or ostium of an aqueous collector channel, to stabilize, maintain patency of, or otherwise improve or stabilize flow through that collector channel. A non-limiting example of such method is described below and shown in the accompanying FIGS. 2 through 4A.

### Step 1: Locate Target Collector Channel(s) for Treatment

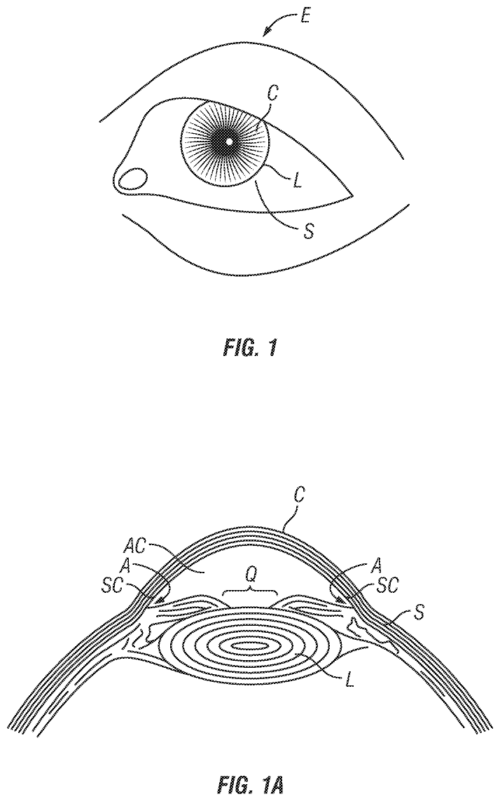

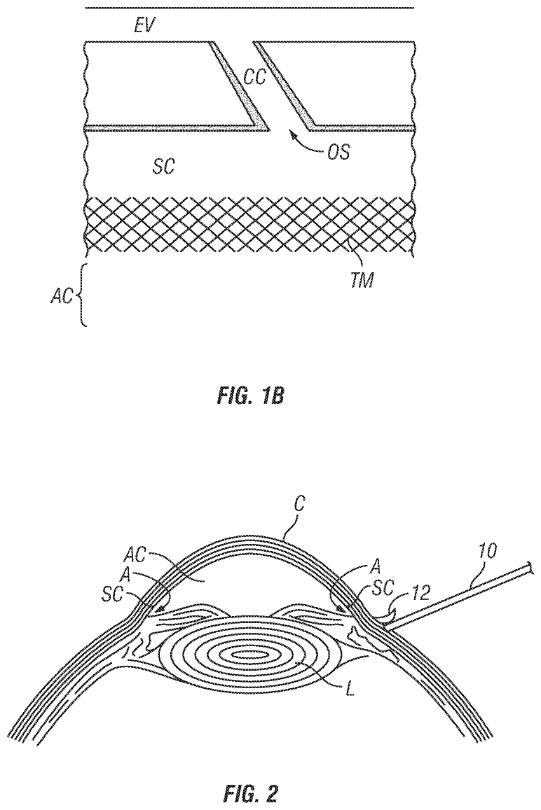

With reference to the accompanying drawings, in Step 1, target collector channels CC are located. This may be accomplished by assessing flow through the collector channels CC and/or their propensity to remain patent or open. Such assessment of flow through or patency of the collector channels CC may be accomplished by direct (e.g., endoscopic) visualization, imaging, and/or any other suitable flow measurement means. One example of a system and method for assessment of regional variations in aqueous outflow though collector channels CC is described in United States Patent Application Publication No. 2016/0262606 entitled Device, System and Method for Functional Imaging of Episcleral Vessels (Mosaed et al.) the entire disclosure of which is expressly incorporated herein by reference. Ms described in Mosaed et al., a contrast agent is introduced into the anterior chamber and an imaging probe, such as a laser microendoscope, is then used to image the contrast agent as it flows outwardly through collector channels in different regions of interest. The imaging probe may be positioned ab interno or ab externo. FIG. 2 shows an example of an ab externo method as described in United States Patent Application Publication No. 2016/0262606, wherein a scleral flap incision 12 is formed and a confocal laser endoscope 10 is positioned under the flap incision 12. Contrast agent is introduced into the anterior chamber AC and, thereafter, Image data from the endoscope is obtained and analyzed to assess functional outflow of the contrast agent through imaged regions of interest. This technique may be used to identify one or more regions in which collector channels CC exhibit desirable flow capacity. In at least some instances, the selected target collector channel(s) will exhibit good outflow capacity and, as shown in FIG. 1B will have openings OS into Schlemm's Canal SC which remain open (patent) so that upon subsequent laser photocoagulation, they can be stabilized of “frozen” in an open state by the laser energy.

### Step 2: Unroof Schlemm's Canal

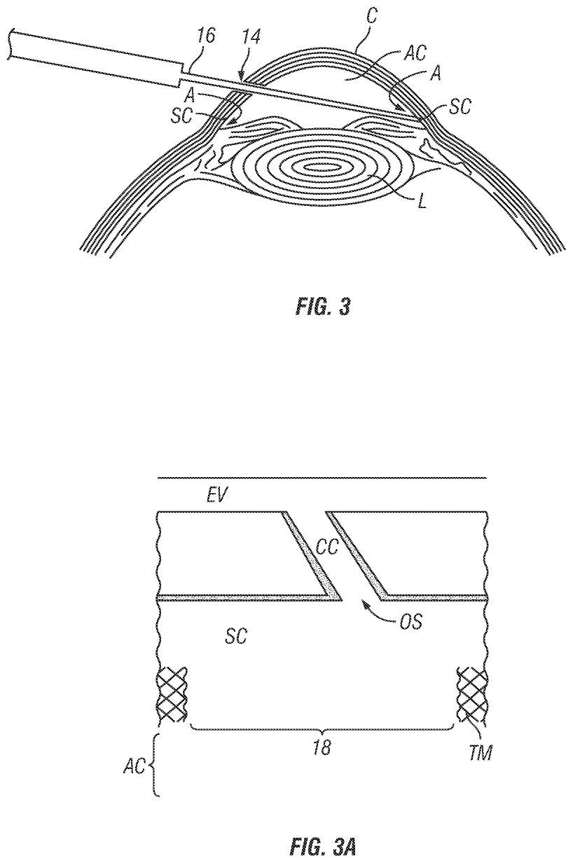

In Step 2, after the target collector channel(s) CC have been identified, segment(s) if trabecular meshwork TM tissue is/are severed, ablated, incised, separated, removed, striped or otherwise opened to thereby un-roof region(s0 of Schlemm's Canal SC in a manner that exposes or provides access to the orifice or ostium of each target collector channel CC. This un-roofing of Schlemm's Canal may be accomplished by any suitable means. FIG. 3 shows one example wherein a device 16, such as a knife, dual blade knife, goniotomy knife, goniotome, cutting apparatus, ablating apparatus, electrosurgical apparatus, cautery, bipolar probe, energy emitting probe, laser probe, TrabEx™ device, TrabEx+™ device, Trabectome® device, Kahook Dual Blade® (KDB or KDB Glide) device, or other suitable apparatus is used to incise, sever, ablate, tear, remove, cut a strip from, or otherwise form one or more opening(s) in the trabecular meshwork tissue TM. Such device 16 may be inserted through a small incision or opening 14 in the cornea C and into the anterior chamber AC, as shown. The distal tip of this device 16 is then advanced into the iridocorneal angle A, adjacent to the target collector channel(s) CC. The device 16 is then used to incise, sever, ablate, remove or form an opening in the trabecular meshwork TM in a manner that exposes of provides access to the orifice(s) OS of the target collector channel(s) CC on the scleral side of Schlemm's Canal SC. Examples of commercially available devices 16 which may be used for this purpose include the TrabEx™, TrabEx+™ and Trabectome® devices available from Microsurgical Technology, Inc., Redmond, Wash. and the Kahook Dual Blade® device available from New World medical, Inc., Rancho Cucamonga, Calif. Alternatively, a laser such as an excimer could be used to deliver laser energy to effect laser ablation of a portion or region of trabecular meshwork TM tissue. Non-limiting examples of a lasers useable to perform this un-roofing step of the process has been reported in probe useable for this purpose have been described in, for example, Durr, G. M., et al; Current review of Excimer laser Trabeculostomy; Eye and Vision; 7, 24 (2020) trabecular meshwork tissue could be accomplished by an ab externo surgical approach, as known in the art.

### Step 3: Position Treatment Delivery Device

After Schlemm's Canal SC has been unroofed, a laser delivery probe 22 is inserted into the anterior chamber AC and positioned to deliver laser treatment to at least the orifice OS of each target collector channel CC, thereby aiming the laser so that the treatment energy is received within the desired collector channel CC. Each collector channel orifice OS is typically 20-50 micron in diameter. In some embodiments, the laser probe 22 may incorporate an endoscope of camera useable to visualize the orifice OS of each target collector channel CC to facilitate aiming of the laser. Alternatively or additionally, the laser delivery probe 22 may incorporate a locator apparatus for mechanically or physically locating the orifice OS of each target collector channel CC, an example of which is shown in Figures &A and 7B and described herebelow. In some instances it may be possible to deliver laser energy to two or more collector channel orifices OS while in other instances the laser probe may be positioned to treat only one collector channel orifice OS at a time. In some instances, treatment of a single collector channel CC will be sufficient. In other instances, two or more collector channels CC will be un-roofed and treated.

### Step 4: Deliver Treatment to Collector Channel

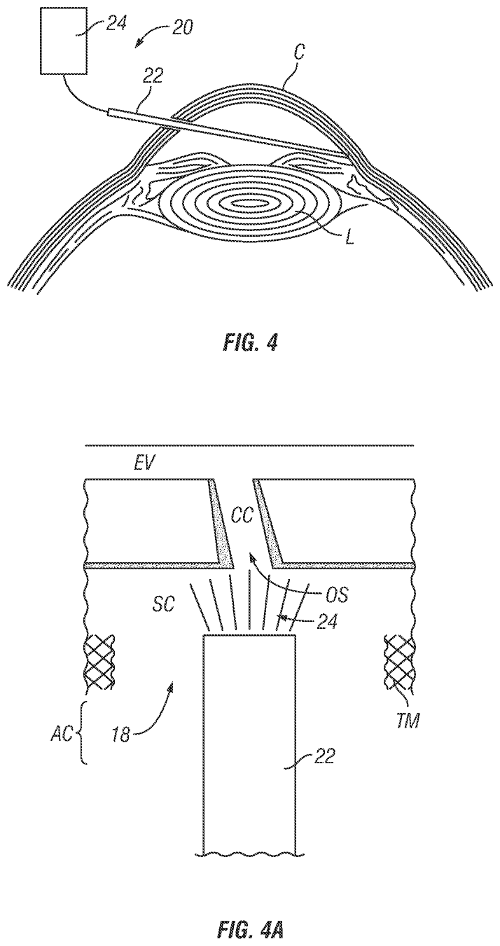

With the laser probe 22 aimed at the target collector channel orifice(s) OS, the laser system 20 is then used to deliver a desired dose of a desired type of laser energy to the target collector channel(s) CC. For example, as illustrated in FIGS. 4 and 4A, the laser probe 22 may incorporate fiber optic wave guide(s) and laser energy may be delivered from a laser generator 24 (e.g., an argon, krypton, diode or frequency doubled ND: YAG laser), through such wave guide to thereby deliver treatment energy 24 from the probe 22 to cause photocoagulation of at least the orifice OS of a target collector channel CC as illustrated in FIG. 4A. This photocoagulation will cause the orifice OS to be “frozen” in a fully open configuration thereby improving aqueous outflow through that orifice OS and the associated collector channel CC. Such improvement of aqueous outflow will reduce intraocular pressure in the eye E. One example of a type of laser that may be used is an argon laser and, for example, each treatment may deliver argon laser energy at 1 watt, non-pulsed, for a duration of 0.5 to 1 second, with a spot size of 100 microns.

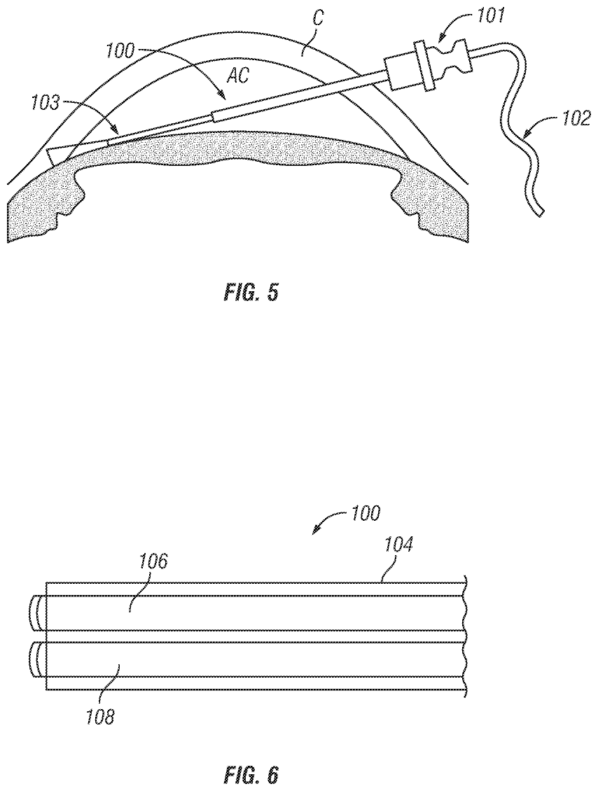

In some embodiments, the laser probe 22 described above may comprise a laser microendoscope 100, an example of which is shown in FIGS. 5 and 6. The laser microendoscope may be used for causing photocoagulation of target collector channel(s) CC in the eye E. The microendoscope device 100 generally includes a handpiece 101, a cable 102, and an intraocular tip 103.

The handpiece 101 comprises an elongate body having a proximal end, a distal end, and at least one lumen running therethrough. Handpiece 101 can comprise any suitable shape, such as rounded shapes and faceted shapes. The shape of handpiece 101 may be described from the shape of its cross section. For example, when viewed head on, handpiece 101 may have a circular cross section, an ovoid cross section, or a polygonal cross section having three, four, or more sides. Handpiece 101 can have any suitable size. In some embodiments, handpiece 101 can have a length between 5 and 10 cm. In some embodiments, handpiece 101 can have a width between 1 and 3 cm. In some embodiments, handpiece 101 can comprise exterior features for improving grip, such as one or more ridges, grooves, bumps, and the like. Handpiece 101 can comprise any suitable material, including but not limited to plastics and metals.

At least one cable 102 extends through the at least one lumen of handpiece 101. The at least one cable 102 carries the signals sent and acquired by the microendoscope device 100. For example, in some embodiments, cable 102 may carry ultrasound signals. In other embodiments, an optic fiber cable 102 may carry optical signals. In certain embodiments, a plurality of cables is provided for carrying a plurality of signals including but not limited to argon, krypton, diode or frequency doubled ND: YAG lasers. In some embodiments, at least one cable 102 may be included to provide power to microendoscope device 100.

Intraocular tip 103 is positioned at the distal end of handpiece 101. In some embodiments, intraocular tip 103 is sized to fit within a microincision in the eye. In some embodiments, the laser emitting microendoscope device enters the anterior chamber under gonioscopic view to reach the angle on the opposite side and laser energy is applied or delivered to the anterior chamber of the eye. This causes enlargement or stabilization of the collector vessels in an eye and reduces IOP.

In certain embodiments, handpiece 101 can be adapted for attachment to existing handpiece systems (such as phacoemulsification handpieces, the Trabectome® handpiece, or the iStent® injector). The microendoscope device is suitable for any procedure, including ab-intemo and ab-externo procedures. An example of ab interno positioning of the device 100 while imaging the outflow collector channels CC is seen in FIG. 5 and an example of such ab externo positioning is seen in FIG. 2 and described above. The dimensions of the device 100 make it suitable for use in imaging anatomical structures of the eye. In some embodiments, the device is capable of imaging aqueous drainage structures of the eye. For example, the device is capable of imaging Schlemm's canal, septations within Schlemm's canal, channels downstream from Schlemm's canal, and the like.

As noted above, in some embodiments, the laser microendoscope device 100 may be useable for both imaging of the collector channels CC as well as for delivering laser energy to form an opening in the trabecular meshwork TM and/or to photocoagulate or otherwise treat the target collector channel(s) CC, ostia OS and or nearby tissues as described herein. In the example of FIG. 6 a laser microendoscope 100 includes both an imaging fiber bundle or waveguide 106 as well as a laser delivery optical fiber bundle 108, so that the mircroendoscope 100 may be used for locating the target collector channel(s) in Step 1 of the above-described method example and for delivering laser energy to accomplish either or both of Steps 3 and 4 of the above-described method example. For example, if laser ablation is the operative modality used to unroof Schlemm's Canal in Step 2 of the method, this single device could be used to accomplish all of Steps 1 through 4. For example, in Step 1, the microendoscope 100 can be used to acquire the images used for identifying the target collector channel(s) as described in. In Step 2, the microendoscope 100 can be used to deliver ablative laser energy to a region of the trabecular meshwork TM thereby ablating or creating an opening in the trabecular meshwork TM thereby exposing or providing access to the target collector channel(s) CC. Any suitable type of laser may be used for ablating or creating an opening in the trabecular meshwork TM in Step 2, such as for example an excimer laser as known for use in performing the excimer laser trabeculotomy procedure described above. In Step 3, the microendoscope 100 could may maneuvered to a position where the laser delivery fibers/waveguide 108 is appropriately aimed at one or more orifice(s) of target collector channels CC. In Step 4, laser energy (e.g., argon laser energy at 1 watt, non-pulsed, for a duration of 0.5 to 1 second, with a spot size of 100 microns) may be delivered through deliver fibers/waveguide 108 to treat the target collector channel(s).

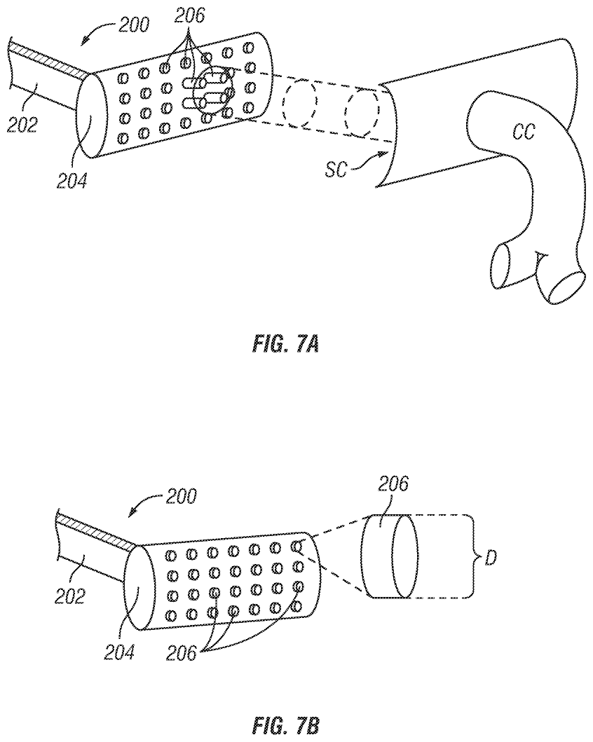

FIG. 7A and 7B show an example of a laser treatment device 200 which incorporates mechanical locators 206 useable to facilitate positioning of the device and delivery of the desired laser treatment to the desired collector channel orifice OS. In general, this device 200 comprises a shaft 202 having a member 204 sized and configured to be advance through the un-roofed region of the trabecular meshwork TM and into Schlemm;s Canal SC. An array of extendable rods 206 which are pressed against the wall of the Schlemm's Canal SC. The rods 206 are then urged forward with sufficient force that the rods 206 which enter the orifice OS of a collector channel CC will advance further than rods 206 that abut against the wall of Schlemm's Canal. Each rod 206 may have a diameter D of, for example, 10 microns. The rods 206 which extend the furthest will thereby indicate the location of the orifice(s) OS of collector channel(s) CC to which treatment energy is to be delivered. In at least some embodiments, the rods 206 may be connected with a laser transducer and laser energy will be delivered through only the rods 206 that have extended into a collector channel CC orifice OS and/or those around its periphery, thereby selectively delivering the laser treatment into the orifice OS of the target collector channel CC and/or to tissue surrounding that orifice OS.

The disclosures of each and every patent, patent application, and publication cited herein are hereby incorporated herein by reference in their entirety.

Although the invention has been described hereabove with reference to certain examples or embodiments of the invention, various additions, deletions, alterations and modifications may be made to those described examples and embodiments without departing from the intended spirit and scope of the invention. For example, any elements, steps, members, components, compositions, reactants, parts or portions of one embodiment or example may be incorporated into or used with another embodiment or example, unless otherwise specified or unless doing so would render that embodiment or example unsuitable for its intended use. Also, where the steps of a method or process have been described or listed in a particular order, the order of such steps may be changed unless otherwise specified or unless doing so would render the method or process unsuitable for its intended purpose. Additionally, the elements, steps, members, components, compositions, reactants, parts or portions of any invention or example described herein may optionally exist or be utilized in the absence or substantial absence of any other element, step, member, component, composition, reactant, part or portion unless otherwise noted. All reasonable additions, deletions, modifications and alterations are to be considered equivalents of the described examples and embodiments and are to be included within the scope of the following claims.

1. A system useable for treating a target aqueous collector channel in an eye of a subject to increase outflow of aqueous humor through that target collector channel, said system comprising:
a first device or system useable to locate the target collector channel;
a second device or system useable to form an opening in trabecular meshwork tissue which overlies or provides access to a portion of a Schlemm's Canal of the eye in which an orifice or ostium of the target collector channel is located;
a third device or system useable to treat tissue in or near the target collector channel to improve patency, openness, size, flow capacity or competency that target collector channel and/or an orifice or ostium associated with that target collector channel.

2. A system according to claim 1 wherein:
the first device or system comprises a scope, imaging apparatus, microendoscope, laser microendoscope or other apparatus operable to image, determine size, patency or flow capacity of, or to locate, said target aqueous collector channel channel(s);
the second device or system comprises a knife, dual blade knife, goniotomy knife, goniotome, cutting apparatus, ablating apparatus, electrosurgical apparatus, cautery, bipolar probe, energy emitting probe, laser probe,
TrabEx™ device, TrabEx+™ device, Trabectome® device, Kahook Dual Blade® device, dual blade knife, or other suitable apparatus to incise, sever, ablate, tear, remove or otherwise form said one or more opening(s) in trabecular meshwork tissue which overlies or is located near target collector channel; and
the third device or system comprises an energy-emitting probe, laser, tissue warming or heating device, coagulation device, or other apparatus operable to photocoagulate, heat, thermally modify, optically modify or otherwise treat tissue in or near target collector channel(s) to improve patency, openness, size, flow capacity or competency of target collector channel(s) and/or orifice(s) or ostia associated with target collector channel.

3. A system according to claim 1 wherein the first device or system, second device or system and third device or system are separate from one another.

4. A system according to claim 1 wherein at least two of said first device or system, second device or system and third device or system comprise a single entity or single device or system.

5. A system according to claim 1 wherein all of said first device or system, second device or system and third device or system comprise a single entity or single device or system.

6. A system according to claim 1 wherein the third device or system comprises a laser of a type selected from; argon, krypton, diode or frequency doubled ND: YAG lasers.

7. A system according to claim 6 wherein the laser delivers argon laser energy at 1 watt, non-pulsed, for a duration of 0.5 to 1 second, with a spot size of 100 microns.

8. A system according to claim 1 wherein the third device or system comprises an apparatus for aiming, aligning or positioning that device or system relative to an orifice or ostium or a target collector channel.

9. A system according to claim 8 wherein the apparatus for aiming, aligning or positioning comprises a scope or imaging device useable to visualize the orifice or ostium of a target collector channel.

10. A system according to claim 8 wherein the apparatus for aiming, aligning or positioning comprises one or more rods or members which advance or insert into an orifice or ostium of a target collector channel.

11. A system according to claim 10 wherein laser energy or another form of energy is deliverable through or about a periphery of said one or more rods or members which advance or insert into an orifice or ostium of a target collector channel.

12. A system according to claim 10 wherein the apparatus for aiming, aligning or positioning comprises:
a head member which is positionable in a Schlemm's Canal of the eye; and
a plurality of said extendable rods or members configured to extend from said head member when said head member is positioned in Schlemm' s Canal;
wherein those extendable rods or members which are aligned with an orifice or ostium of a collector channel will extend further than others of said extendable rods or members.

13. A system according to claim 12 wherein the third device or system is further configured to selectively deliver laser energy or other treatment through those extendable rods or members which have extended further than others of said extendable rods or members, thereby selectively delivering laser energy or other treatment to or through an orifice or ostium of a collector channel into which those extendable rods or members have advanced.

14. A system according to claim 12 wherein the third device or system is further configured to selectively deliver laser energy or other treatment to an area around or near those extendable rods or members which have extended further than others of said extendable rods or members, thereby selectively delivering laser energy or other treatment to an area that is around or near an orifice or ostium of a collector channel.

15. A method for using a system according to claim 1 to improve or stabilize drainage of aqueous humor from an anterior chamber of an eye of a subject, said method comprising the steps of:
a) using the first device or system to locate the target aqueous collector channel;
b) using the second device or system to form one or more opening(s) in trabecular meshwork tissue to un-roof one or more region(s) of Schlemm's Canal of the eye in a manner which exposes or allows access to the target collector channel; and
c) using the third device or system to treat an orifice or ostium of the target collector channel and/or an area around or near the orifice or ostium and/or a wall or portion of a wall of the target collector channel, in a manner that increases outflow of aqueous humor from an anterior chamber of the eye through the target collector channel.

16. (canceled)

17. (cancelled)

18. (cancelled)

19. (canceled)

20. (cancelled)

21. A method according to claim 15 wherein the step of treating the orifice or ostium of the target collector channel and/or an area around or near the orifice or ostium and/or a wall or portion of a wall of the target collector channel in a manner that increases outflow of aqueous humor from an anterior chamber of the eye through the target collector channel comprises:
delivering laser or other energy to photocoagulate, stiffen, reduce flexibility of, reduce elasticity of, or otherwise modify the orifice or ostium and/or tissue around or near the orifice of ostium and/or a wall or portion of a wall of the target collector channel in a manner that reduces likelihood of collapse or close of, maintain patency of and/or improves flow through the orifice or ostium and/or the collector channel.

22. (canceled)

23. (canceled)

24. A method according to claim 15 wherein the third device or system effects said treatment by delivering laser or other treatment energy.

25. A method according to claim 24 wherein the third device or system further comprises a scope, imaging apparatus or other visualizing apparatus which is used to aim, align or position the third device before delivering said laser or other treatment energy.

26. A method according to claim 24 wherein the third device further comprises one or more rods or members which advance or insert into the orifice or ostium of the target collector channel and wherein the laser energy or other treatment energy is delivered through or about a periphery of said one or more rods or members channel in a manner that increases outflow of aqueous humor from an anterior chamber of the eye through the target collector channel.

27. A method according to claim 15 wherein the second device or system forms said one or more opening(s) in trabecular meshwork tissue by delivering excimer laser energy and the third device or system effects said treatment by delivering argon laser energy.
